SEQUENCE LISTING (24 sequences)

SEQ ID NO: 1 - protein, 398 aa, Homo sapiens
NPASPPEEGSPDPDSTGALVEEEDPFFKVPVNKLAAAVSNFGYDLYRVRSSMSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 2 - protein, 11 aa, Artificial sequence
  Synthetic peptide
TGALVEEEDPF

SEQ ID NO: 3 - protein, 11 aa, Artificial sequence
  Synthetic peptide
ERTESIIHRAL

SEQ ID NO: 4 - protein, 17 aa, Artificial sequence
  Synthetic peptide
ERTESIIHRALYYDLIS

SEQ ID NO: 5 - protein, 11 aa, Artificial sequence
  Synthetic peptide
SSPDIHGTYKE

SEQ ID NO: 6 - protein, 17 aa, Artificial sequence
  Synthetic peptide
LYYDLISSPDIHGTYKE

SEQ ID NO: 7 - protein, 5 aa, Artificial sequence
  Synthetic peptide
VEEDP

SEQ ID NO: 8 - protein, 11 aa, Artificial sequence
  Synthetic peptide
TGALVQQQDPF

SEQ ID NO: 9 - protein, 18 aa, Artificial sequence
  Synthetic peptide
TGALVEEEDPFFKVPVNK

SEQ ID NO: 10 - protein, 10 aa, Artificial sequence
  Synthetic peptide
TGASSEEEDP

SEQ ID NO: 11 - protein, 25 aa, Artificial sequence
  Synthetic peptide
PVNKLAAAVSNFGYDLYRVRSSMSP

SEQ ID NO: 12 - protein, 25 aa, Artificial sequence
  Synthetic peptide
PVNKLAAAVSNFGYNLYRVRSSMSP

SEQ ID NO: 13 - protein, 6 aa, Artificial sequence
  Synthetic peptide
KVPVNK

SEQ ID NO: 14 - protein, 6 aa, Artificial sequence
  Synthetic peptide
SNFGYD

SEQ ID NO: 15 - protein, 9 aa, Artificial sequence
  Synthetic peptide
YRVRSSMSP

SEQ ID NO: 16 - protein, 6 aa, Artificial sequence
  Synthetic peptide
DERTES

SEQ ID NO: 17 - protein, 7 aa, Artificial sequence
  Synthetic peptide
HRALYYD

SEQ ID NO: 18 - protein, 6 aa, Artificial sequence
  Synthetic peptide
YYDLIS

SEQ ID NO: 19 - protein, 17 aa, Artificial sequence
  Synthetic peptide
ERTESIIHRALYYNLIS

SEQ ID NO: 20 - protein, 17 aa, Artificial sequence
  Synthetic peptide
ERTESSSHRALYYDSSS

SEQ ID NO: 21 - protein, 17 aa, Artificial sequence
  Synthetic peptide
QRTQSIIHRALYYNLIS

SEQ ID NO: 22 - protein, 30 aa, Artificial sequence
  Synthetic peptide
ERTESIIHRALYYDLISSPDIHGTYKELLD

SEQ ID NO: 23 - protein, 32 aa, Artificial sequence
  Synthetic peptide
DERTESIIHRALYYDNNKVPVNKLAAAVSNFG

SEQ ID NO: 24 - protein, 6 aa, Artificial sequence
  Synthetic peptide
TQVEHR

(19) United States
(12) Patent Application Publication
(10) Pub. No.: US 2006/0189519 A1
(43) Pub. Date: Aug. 24, 2006

(54) Anti-angiogenic fragments fo pigment epithelium-derived factor (pedf)

(76) Inventors: Karl Volz, Oak Park, IL (US); Stephanie Filleur, Chicago, IL (US); Olga Volpert, Wilmette, IL (US); Tetiana Zaichuk, Oak Park, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 10/529,510

(22) PCT Filed: Sep. 26, 2003
(86) PCT No.: PCT/US03/30264

§ 371 Date: Oct. 12, 2005

Related U.S. Application Data
(60) Provisional application No. 60/413,685, filed on Sep. 26, 2002.
(60) Provisional application No. 60/417,688, filed on Oct. 10, 2002.

(51) Int. Cl. A61K 38/18; C07K 14/485

(52) U.S. Cl. 514/12; 530/399

(57) ABSTRACT

The present invention provides anti-angiogenic derived from pigment epithelium-derived factor (PEDF) pharmaceutical compositions comprising the peptides, and methods of preventing angiogenesis. Such methods are useful in treating angiogenesis-associated disorders and diseases.

### RELATED APPLICATION DATA

This application claims priority to U.S. provisional application Ser. No. 60/413,685, filed on Sep. 26, 2002, and U.S. provisional application Ser. No. 60/417,688, filed on Oct. 10, 2002, the disclosures of which are incorporated by reference in their entirety.

### BACKGROUND

Diabetes mellitus, a hyperglycemic condition due to improper production and/or utilization of insulin, afflicts 6% of the United States population, and results in 200,000 deaths every year. Two of the most serious complications of diabetes are kidney failure and loss of vision. Both blindness and the end-stage renal disease involve major vascular abnormalities.

Diabetic retinopathy is a progressive eye disease, starting with the damage to the small vessels in the retina. Two vascular layers in the posterior eye—the capillaries of the choroid, and the vessels in the retinal bed adjacent to the vitreous—are highly regulated and compartmentalized, and in normal adult eye these vessels remain quiescent. However, during the later stages of retinopathy, a decrease in blood supply is often followed by neovascularization. Vascular endothelial growth factor (VEGF), the main angiogenic stimulus involved in ischemic retinopathy, also causes fenestration and leakiness of the new and pre-existing vasculature. The invasion of the expanding capillaries into the vitreous humor (proliferative retinopathy) often leads to hemorrhage, scarring, and retinal detachment. Currently, this end condition is irreversible, with little to no treatment options available. Low oxygen tension caused by ischemia of the retinal vessels is a strong positive regulator of VEGF production. (Shweiki et al., 1995 and Pe'er et al., 1996).

Angiogenesis, the sprouting of new capillaries from pre-existing vasculature, is tightly suppressed in the healthy adult eye. In the healthy eye, the angiostatic state results from a balance between multiple endogenous angiogenic stimuli and inhibitors. One of the key inhibitors of ocular neovascularization is pigment epithelium-derived factor (PEDF), a protein present in the vitreous fluid and cornea. PEDF's function is twofold: while suppressing neovascularization, it maintains the viability of neuronal cells in the eye through its neurotrophic activity.

Although numerous growth factors, cytokines, and inhibitors of angiogenesis have been found in the eye, only two factors are influenced by oxygen levels: VEGF, and pigment epithelium-derived growth factor (PEDF). (Casey et al., 1997). PEDF, secreted by the retinal pigment epithelium (RPE) cells in high concentrations, is thought to be the major inhibitor of angiogenesis, thus responsible for the angiostatic state of the adult eye. (Dawson et al., 1999A) While VEGF production is suppressed at high O₂ levels and promoted by hypoxia, PEDF is regulated in an opposite manner, remaining high in normoxia and decreasing under hypoxia. (Dawson et al., 1999A and Aiello et al., 1994) A number of studies done with animal models of diabetic retinopathy and retinopathy of prematurity show that the course of retinopathy following ischemia is not determined by VEGF alone, but rather by the ratio between pro-angiogenic VEGF and anti-angiogenic PEDF. (Gao et al. 2002, and Ohno-Matsui et al., 2001)

PEDF was first identified as an anti-angiogenic factor secreted by the retinoblastoma cells and responsible for the anti-angiogenic state and light transmission through the cornea and vitreous. (Dawson et al., 1999A) PEDF is a highly potent anti-angiogenic factor active against wide variety of angiogenic stimuli with specific activity close to or higher than that of thrombospondin-1, angiostatin and endostatin. (Dawson et al., 1999A) It was also shown that PEDF acts to block angiogenesis by specifically inducing endothelial cell apoptosis via secondary receptor-mediated cascade involving CD95/Fas receptor and its ligand FasL. (Volpert et al., 2002)

The information on the PEDF receptor responsible for its anti-angiogenic activity is limited. PEDF's anti-angiogenic activity was shown to be dose-dependent. (Stellmach et al., 2001) The receptor was speculated to be different than the neurotrophic receptor. (Stellmach et al., 2001)

### SUMMARY

In one aspect, the present invention provides an anti-angiogenic pigment epithelium-derived factor (PEDF) fragment or analog thereof. Preferably, the anti-angiogenic peptide contains 5-50 contiguous amino acids of SEQ ID NO:1, such as TGALVEEEDPF (TGA), ERTESIIHRALYYDLIS (ERT-L), and DPFFKVPVNKLAAAVSNFGYDLYRVRSSMSPTTN (34-mer). One or more terminus of the peptide can be altered. Furthermore, the peptide can be part of a pharmaceutical composition further comprising a buffer or excipient.

In another aspect, the present invention provides a method of inhibiting endothelial cell migration or proliferation. Such method comprises contacting an endothelial cell, in vitro or in vivo, with a pharmaceutical composition comprising an effective amount of a PEDF peptide fragment or analog thereof having anti-angiogenic activity. Such methods are particularly useful when an anti-angiogenic amount of the peptide is administered to a patient with a disease or disorder associated with neovascularization, such as an ophthalmologic disease or disorder or a malignant or metastatic condition.

In another aspect, the present invention provides for the use of an anti-angiogenic PEDF fragment or analog thereof in the preparation of a medicament for treating cancer or an opthalmological disease or disorder.

The present invention further provides kits and medical devices comprising an anti-angiogenic PEDF fragment or analog. Such kits and medical devices are useful in methods of treating cancer or an opthalmological disease or disorder.

In another aspect, the present invention provides a method of predicting whether a diabetic patient will develop proliferative retinopathy comprising determining the ratio of vascular endothelial growth factor (VEGF) to PEDF in an ocular fluid sample from said patient.

In yet another aspect, the present invention provides an anti-angiogenic PEDF fragment analog comprising one or more amino acid insertions, deletions, or substitutions to a PEDF fragment.

### BRIEF DESCRIPTION OF THE FIGURES

FIG. 1. VEGF and PEDF levels in diabetic patients with and without proliferative retinopathy. VEGF was measured in anterior fluid samples by ELISA (R&D Systems VEGF assay kit) and PEDF using semi-quantitative Western blotting/densitometry analysis. Horizontal dashed lines indicate the average VEGF levels. Vertical dashed lines indicate average levels of PEDF. FIG. 1A Normal controls (cataract only); FIG. 1B Diabetics that did not progress to proliferative retinopathy during 5-year follow-up period; FIG. 1C Patients that developed retinopathy within 5 years since diagnosis.

FIG. 2. In vitro angiogenic activity of the ocular fluids from normal and diabetic patients. Angiogenic activity was measured in the endothelial cell migration/chemotaxis assay. FIG. 2A Anterior chamber fluids from normal (empty bars), diabetic, −PR (gray bars) and diabetic, +PR (black bars) donors. FIG. 2B Fluids that were non-angiogenic were tested in combination with VEGF (empty bars) or with VEGF and neutralizing anti-PEDF antibodies (hatched bars).

FIG. 3. In vitro anti-angiogenic activity by 33-mer and 44-mer PEDF peptides. Microvascular endothelial cells (HMVECs) chemotaxis up the gradient of pro-angiogenic VEGF was examined in the presence of the 44-mer or 34-mer. FIG. 3A Recombinant PEDF (rPEDF, BH) (10 nM), 34-mer (1 μM) and 44-mer (1 μM) were tested alone (empty bars), or in the presence of VEGF (hatched bars). Anti-PEDF neutralizing antibodies were added where shown (filled bars). FIG. 3B 34-mer (●) and 44-mer (♦) were tested with VEGF (100 pg/ml) at increasing concentrations. ED₅₀ was determined using regression curves.

FIG. 4. The first generation of PEDF peptides synthesized and tested for anti-angiogenic activity. There are two representations for each peptide. The bottom row shows the peptides as shaded ribbons relative to the α-carbon backbone of the rest of the molecule, while the top row shows in dark shading the solvent accessible surfaces of the peptides in the context of the rest of the molecule. From left to right, the peptides are from the following components of PEDF: 1) amino terminal loop and helix, 2) hC, 3) hC plus loop, 4) hD, and 5) loop plus hD.

FIG. 5. Endothelial cell apoptosis induced by TGA and ERT-L PEDF peptides. 90% confluent HUVECs were treated with increasing concentrations of TGA (FIG. 5A) or ERT-L (FIG. 5B). Apoptotic cells were detected with ApopTag assay kit (Intergen) and percent of TUNEL positive cells calculated.

FIG. 6. Linear diagram of active peptides from PEDF's amino terminus that showed anti-angiogenic activity. PEDF's primary sequence from residue 16 to 101. Every tenth residue is labeled with a dot, and the secondary structural elements are shown above the dots. The four peptides discussed in the Esamples are shown by brackets.

FIG. 7. Stereo diagram of relative positions of the amino terminus and α-helices hA and hC. Note the proximity of the C-terminus of hC to the N-terminus of hA. Design of a short peptide linker (arrow) would combine the two helices into the new sequence hC-linker-hA, but would still retain their relative spatial positions and all of the functional groups of the N-term, hA, and hC. This same approach is feasible for other secondary structural units in the putative signaling region.

### DETAILED DESCRIPTION OF PREFERRED EMBODIMENTS OF THE INVENTION

PEDF, a 50 kDa protein and inhibitor of angiogenesis, is abundantly expressed in the eye. PEDF is the chief factor responsible for the maintenance of angiostasis, necessary to retain clarity of the light-transmitting components of the eye. PEDF is also a neurotrophic factor that promotes survival and differentiation of retinal pigment epithelial cells (RPE), also contributing to normal vision.

The present invention relates to anti-angiogenic methods and compositions based on fragments of Pigment epithelium-derived factor (PEDF). The invention provides for treatment of neovascular disorders by administration of a composition comprising an anti-angiogenic compound of the invention. Such compounds include PEDF fragments and analogs thereof. In some embodiments, the invention provides treatment of an ocular disorder associated with neovascularization. In other embodiments, the invention provides a treatment of a cancerous condition or prevents progression from a pre-neoplastic or non-malignant state into a neoplastic or malignant state.

Anti-Angiogenic PEDF Fragments and Analogs Thereof

The amino acid sequence of human PEDF (SEQ ID NO:1) is known in the art. (Siminovic et al., 2001) Although anti-angiogenic fragments, analogs, and mimics of human PEDF are preferred, such molecules derived from other mammalian PEDF are within the scope of the invention. Examples of other mammalian PEDF polypeptides are mouse (GenBank Acc. No. P97298) and bovine (GenBank Acc. No. Q95121).

The invention provides anti-angiogenic fragments of PEDF. By “fragment,” it is meant that the peptide comprises only a portion of the amino acid sequence of PEDF (SEQ ID NO:1). Anti-angiogenic activity may be measured in a number of ways. Examples of in vitro and in vivo assays for angiogenic activity include endothelial cell migration assay, endothelial cell apoptosis assay, JNK-1 kinase assay, mouse corneal neovascularization assay, chick chorioallantoic membrane assay, and rabbit corneal pocket assay.

The invention provides for PEDF fragments or analogs thereof consisting of or comprising at least 5 contiguous amino acids of PEDF and having anti-angiogenic activity. In preferred embodiments, the molecule comprises at least 10, at least 20, at least 50, at least 75, at least 100, at least 150, at least 200, or at least 250 contiguous amino acids of PEDF and has anti-angiogenic activity. In some embodiments, the molecule consists essentially of 5, 10, 15, 20, 25, 30, 35, 40, 45, 50, 60, 70, 80, 90, 100, 125, 150, 175, 200, 250, 300, or 350 contiguous amino acids of PEDF. Preferred embodiments include molecules consisting essentially of 5 to 50 contiguous amino acids of PEDF. By “consisting essentially of,” it is meant that the molecules can contain additional modifications to the peptide, e.g., acetyl groups, amide groups, or heterologous amino acids or amino acid sequences, provided such molecules retain angiogenic activity.

Preferred PEDF fragments correspond one or more portions with the amino terminal half of PEDF. More preferred fragments correspond to one or more portions of the first 100 amino acids of PEDF. Examples of such peptides are the 34-mer (amino acids 24-57 of SEQ ID NO:1), the TGA peptide (16-26 of SEQ ID NO:1), and the ERT-L peptide (amino acids 78-94 of SEQ ID NO:1).

In certain embodiments, the invention encompasses anti-angiogenic peptides that are homologous to human PEDF (SEQ ID NO:1) fragments. In some embodiments, the amino acid sequence of the peptide has at least 80% identity with an anti-angiogenic PEDF fragment. In other embodiments, this identity is greater than 85%, 90%, or 95%.

PEDF fragment analogs can be made by altering PEDF sequences by substitutions, additions or deletions. These include, as a primary amino acid sequence, all or part of the amino acid sequence of a PEDF fragment including altered sequences in which functionally equivalent amino acid residues are substituted for residues within the sequence resulting in a silent change. For example, one or more amino acid residues within the sequence can be substituted by another amino acid of a similar polarity which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Alternatively, a non-conservative substitution may be made in an amino aicd that does not constribute to the anti-angiogenic activity of the fragment. Anti-angiogenic activity of an analog can be tested using the assays described herein.

The PEDF fragments and analogs of the invention can be produced by various methods known in the art, including recombinant production or synthetic production. Recombinant production may be achieved by the use of a nucleic acid encoding the sequence of the fragment or analog operably linked to a promoter for the expression of the nucleic acid and optionally a regulator of the promoter. This construct can be placed in a vector, such as a plasmid, virus or phage vector. The vector may be used to transfect or transform a host cell, e.g., a bacterial, yeast, insect, or mammalian cell.

A vector encoding a PEDF fragment or analog thereof having anti-angiogenic activity, along with a host cell comprising such vector, form additional aspects of the present invention.

Synthetic production of peptides is well known in the art and is available commercially from a variety of companies. A peptide corresponding to a portion of a fragment of PEDF which mediates anti-angiogenic activity can be synthesized by use of a peptide synthesizer. Furthermore, if desired, non-classical amino acids or chemical amino acid analogs can be introduced as a substitution or addition into the PEDF fragment sequence. Non-classical amino acids include, but are not limited to, the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, and Nα-methyl amino acids.

Included within the scope of the invention are PEDF fragments or analogs that are differentially modified during or after translation (or synthesis), e.g., by biotinylation, acetylation, phosphorylation, carboxylation, amidation, derivatization by known protecting/blocking groups, proteolytic cleavage, linkage to an antibody molecule or other cellular ligand, etc. Any of numerous chemical modifications ma be carried out by known techniques, including, but not limited to, specific chemical cleavage by cyanogen bromide, trypsin, chymotrypsin, papain, V8 protease, NaBH₄, acetylation, formulation, oxidation, reduction, etc.

In further embodiments, the invention encompasses a chimeric, or fusion, protein comprising a PEDF fragment or analog thereof joined at its amino or carboxy-terminus via a peptide bond to an amino acid sequence of a different protein. Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer.

Peptides of the invention that are of a size suitable for synthetic production can also be made using D-amino acids. In such cases, the amino acids will be linked in reverse sequence in the C to N orientation. This is conventional in the art for producing such peptides.

Anti-Angiogenic Assays of PEDF Fragments and Analogs Thereof

The functional activity and/or therapeutically effective dose of PEDF fragments and analogs can be assayed in vitro and in vivo by various methods. These methods are based on the physiological processes involved in angiogenesis and while they are within the scope of the invention, they are not intended to limit the methods by which PEDF fragments and analogs inhibiting angiogenesis are defined and/or a therapeutically effective dosage of the pharmaceutical composition is determined.

In vitro methods include, but are not limited to, endothelial cell migration and apoptosis assays and JNK-1 kinase activity assays as described in the examples. In vivo methods include, but are not limited to, mouse corneal neovascularization assay, chick chorioallantoic membrane assay, and rabbit corneal pocket assay. Such assays are particularly useful in methods of determining anti-angiogenic activity of a PEDF fragment homolog or analog.

Uses of PEDF Fragments and Analogs Thereof

The invention provides a number of useful methods related to the anti-angiogenic activity of a PEDF fragment or analog thereof. One such method is a method of inhibiting angiogenesis. In such method, a vascular cell, such as an endothelial cell, is contacted with a PEDF fragment or analog thereof. In certain embodiments, the cell is analyzed for one or more characteristics indicative of an anti-angiogenic or angiogenic agent, such as cell migration, lack of proliferation, or apoptosis. In other embodiments, a patient is observed for indication of anti-angiogenic or angiogenic activity (e.g., blood vessel growth or tumor growth) subsequent to the administration of a PEDF fragment or analog thereof.

The invention provides for treatment of diseases or disorders, particularly diseases or disorders associated with neovacsularization. Methods of treatment comprise administering a therapeutically effective amount of an anti-angiogenic PEDF fragment or analog thereof to a patient in need thereof. Patients in need thereof may suffer from one or more disease or disorder associated with neovascularization or may have been determined to have a greater susceptibility to a disease or disorder associated with neovascularization. Thus, treatment includes both therapeutic and prophylactic utility.

Neovascular disease and disorders that can be treated with anti-angiogenic peptides are disclosed in U.S. Pat. No. 6,403,558 (incorporated herein by reference in its entirety).

PEDF mRNA has been detected in most tissues, (Tombran-Tink et al., 1996) suggesting that its anti-angiogenic function may be significant in other organs. On the other hand, serpins other than PEDF have recently been shown to block vessel formation and induce tumor regression. Consequently, the methods, models, and compositions described herein for PEDF may be applied to the structural investigation into the anti-angiogenic functions of other serpin molecules.

Malignant and metastatic conditions which can be treated with an anti-angiogenic PEDF fragment or analog thereof include, but are not limited to, the solid tumors listed below:

Solid tumors

sarcomas and carcinomas

fibrosarcoma

myxosarcoma

liposarcoma

chondrosarcoma

osteogenic sarcoma

chordoma

angiosarcoma

endotheliosarcoma

lymphangiosarcoma

lymphangioendotheliosarcoma

synovioma

mesothelioma

Ewing's tumor

leiomydsarcoma

rhabdomyosarcoma

colon carcinoma

pancreatic cancer

breast cancer

ovarian cancer

prostate cancer

squamous cell carcinoma

basal cell carcinoma

adenocarcinoma

sweat gland carcinoma

sebaceous gland carcinoma

papillary carcinoma

papillary adenocarcinomas

cystadenocarcinoma

medullary carcinoma

bronchogenic carcinoma

renal cell carcinoma

hepatoma

bile duct carcinoma

choriocarcinoma

seminoma

embryonal carcinoma

Wilms' tumor

cervical cancer

testicular tumor

lung carcinoma

small cell lung carcinoma

bladder carcinoma

epithelial carcinoma

glioma

astrocytoma

medulloblastoma

craniopharyngioma

ependymoma

Kaposi's sarcoma

pinealoma

hemangioblastoma

acoustic neuroma

oligodendroglioma

menangioma

melanoma

neuroblastoma

retinoblastoma

Purified PEDF has been successfully used to treat ocular neovascularization. (Stellmach et al., 2001; Chader, G. 2001; and Mori et al., 2001) Described herein are PEDF fragments and agonists that have the ability to inhibit retinal neovascularization, providing for the treatment and prevention of eye disease. Ocular disorders associated with neovascularization which can be treated an anti-angiogenic PEDF fragment or analog thereof include, but are not limited to:

neovascular glaucoma

diabetic retinopathy

retinoblastoma

retrolental fibroplasias

uveitis

retinopathy of prematurity

macular degeneration

corneal graft neovascularization

as well as other eye inflammatory diseases, ocular tumors and diseases associated with choroidal or iris neovascularization.

Other disorders which can be treated with an anti-angiogenic PEDF fragment or analog thereof include, but are not limited to, hemangioma, arthritis, psoriasis, angiofibroma, atherosclerotic plaques, delayed wound healing, granulations, hemophilic joints, hypertrophic scars, nonunion fractures, Osler-Weber syndrome, pyogenic granuloma, scleroderma, trachoma, and vascular adhesions.

An anti-angiogenic PEDF fragment or analog thereof can be tested in vivo for the desired therapeutic or prophylactic activity as well as for determination of therapeutically effective dosage. For example, such compounds can be tested in suitable animal model systems prior to testing in humans, including, but not limited to, rats, mice, chicken, cows, monkeys, rabbits, etc. For in vivo testing, prior to administration to humans, any animal model system known in the art may be used.

Therapeutic and Prophylactic Administration and Compositions for Use Thereof

The invention provides methods of treatment (and prophylaxis) by administration to a subject an effective amount of an anti-angiogenic PEDF fragment or analog thereof. In a preferred aspect, an anti-angiogenic PEDF fragment or analog thereof is substantially purified as set forth in the Examples. The subject is preferably an animal, including, but not limited to, animals such as cows, pigs, chickens, etc., and is preferably a mammal, and most preferably human.

The invention further provides methods of treatment by administration to a subject, an effective amount of an anti-angiogenic PEDF fragment or analog thereof combined with a chemotherapeutic agent and/or radioactive isotope exposure.

The invention also provides for methods of treatment for patients who have entered a remission in order to maintain a dormant state.

Various delivery systems are known and can be used to administer an anti-angiogenic PEDF fragment or analog thereof, e.g., encapsulation in liposomes, microparticles, microcapsules, receptor-mediated endocytosis (see, e.g., Wu and Wu, 1987, J. Biol. Chem. 262:4429-4432). Methods of introduction include, but are not limited to, topical, intradermal, intramuscular, intraperitoneal, intravenous, subcutaneous, intranasal, epidural, ophthalmic, and oral routes. The compounds may be administered by any convenient route, for example by infusion or bolus injection, by absorption through epithelial or mucocutaneous linings (e.g., oral mucosa, rectal and intestinal mucosa, etc.) and may be administered together with other biologically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection e.g., via a syringe, at the site (or former site) of a malignant tumor or neoplastic or pre-neoplastic tissue.

For topical application, an anti-angiogenic PEDF fragment or analog thereof can be combined with a carrier so that an effective dosage is delivered, based on the desired activity (i.e., ranging from an effective dosage, for example, of 1.0 μM to 1.0 mM to prevent localized angiogenesis, endothelial cell migration, and/or inhibition of capillary endothelial cell proliferation. In one embodiment, an anti-angiogenic PEDF fragment or analog thereof is applied to the skin for treatment of diseases such as psoriasis. The carrier may in the form of, for example, and not by way of limitation, an ointment, cream, gel, paste, foam, aerosol, suppository, pad or gelled stick.

A topical composition for treatment of some of the eye disorders comprises an effective amount of an anti-angiogenic PEDF fragment or analog thereof in a ophthalmologically acceptable excipient such as buffered saline, mineral oil, vegetable oils such as corn or arachis oil, petroleum jelly, Miglyol 182, alcohol solutions, or liposomes or liposome-like products. Any of these compositions may also include preservatives, antioxidants, antibiotics, immunosuppressants, and other biologically or pharmaceutically effective agents which do not exert a detrimental effect on the anti-angiogenic PEDF fragment or analog thereof.

For directed internal topical applications, for example for treatment of ulcers or hemorrhoids, a composition may be in the form of tablets or capsules, which can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; or a glidant such as colloidal silicon dioxide. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

Suppositories generally contain active ingredient in the range of 0.5% to 10% by weight; oral formulations preferably contain 10% to 95% active ingredient.

In another embodiment, an anti-angiogenic PEDF fragment or analog thereof can be delivered in a vesicle, in particular a liposome. See, Langer et al., 1990, Science 249:1527-1533; Treat et al., 1989, in Liposomes in the Therapy of Infectious Disease and Cancer, Lopez-Berestein and Fidler (eds.), Liss, New York, pp. 353-365; Lopez-Berestein, ibid., pp. 317-327.

In yet another embodiment, an anti-angiogenic PEDF fragment or analog thereof can be delivered in a controlled release system. In one embodiment, an infusion pump may be used to administer an anti-angiogenic PEDF fragment or analog thereof, such as for example, that are used for delivering insulin or chemotherapy to specific organs or tumors (see Langer, supra; Sefton, CRC Crit. Ref. Biomed., 1987, Eng. 14:201; Buchwald et al., 1980, Surgery 88:507; Saudek et al., 1989, N. Engl. J. Med. 321:574).

In a preferred form, an anti-angiogenic PEDF fragment or analog thereof is administered in combination with a biodegradable, biocompatible polymeric implant which releases the anti-angiogenic PEDF fragment or analog thereof over a controlled period of time at a selected site. Examples of preferred polymeric materials include polyanhydrides, polyorthoesters, polyglycolic acid, polylactic acid, polyethylene vinyl acetate, and copolymers and blends thereof. See, Medical Applications of Controlled Release, Langer and Wise (eds.), 1974, CRC Pres., Boca Raton, Fla.; Controlled Drug Bioavailability, Drug Product Design and Performance, Smolen and Ball (eds.), 1984, Wiley, New York; Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105. In yet another embodiment, a controlled release system can be placed in proximity of the therapeutic target, i.e., the brain, thus requiring only a fraction of the systemic dose (see, e.g., Goodson, in Medical Applications of Controlled Release, 1989, supra, vol. 2, pp. 115-138). Other controlled release systems are discussed in the review by Langer (1990, Science 249:1527-1533).

The present invention also provides pharmaceutical compositions. Such compositions comprise a therapeutically effective amount of an anti-angiogenic PEDF fragment or analog thereof, and a pharmaceutically acceptable carrier.

The pharmaceutical compositions of the invention can be formulated as neutral or salt forms. Pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. The parental preparation can be enclosed in ampoules, disposable syringes or multiple dose vials made of glass or plastic.

These compositions can take the form of solutions, suspensions, emulsion, tablets, pills, capsules, powders, sustained-release formulations and the like. The composition can be formulated as a suppository, with traditional binders and carriers such as triglycerides, microcrystalline cellulose, gum tragacanth or gelatin. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of an anti-angiogenic PEDF fragment or analog thereof, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle, containing sterile pharmaceutical grade water or saline. Where the composition is administered by injection, an ampoule of sterile water for injection or saline can be provided so that the ingredients may be mixed prior to administration.

The amount of the anti-angiogenic PEDF fragment or analog thereof which will be effective in the treatment of a particular disorder or condition will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. However, suitable dosage ranges for intravenous administration are generally about 20-500 micrograms of active compound per kilogram body weight. Suitable dosage ranges for intranasal administration are generally about 0.01 pg/kg body weight to 1 mg/kg body weight. Effective doses may be extrapolated from dose-response curves derived from in vitro or animal model test bioassays or systems.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

### EXAMPLES

### Example 1

### PEDF/VEGF Ratio in Vitreous Fluids of Diabetic Patients is Predictive of Disease Outcome

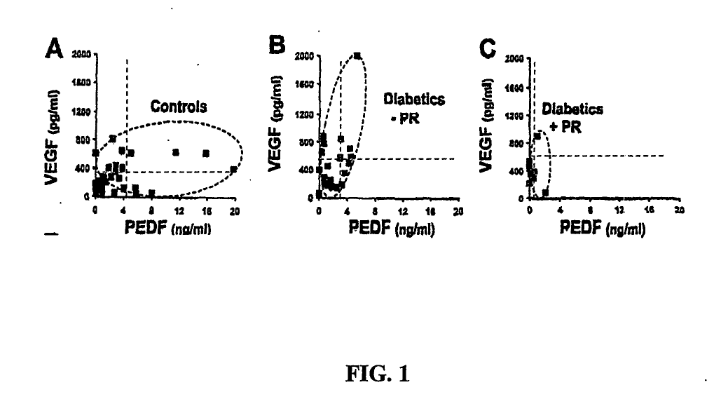

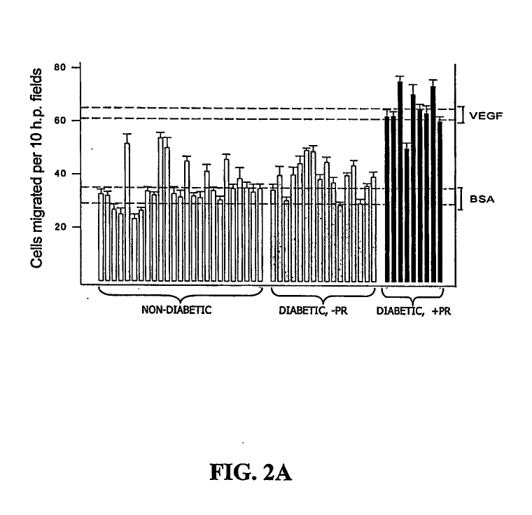

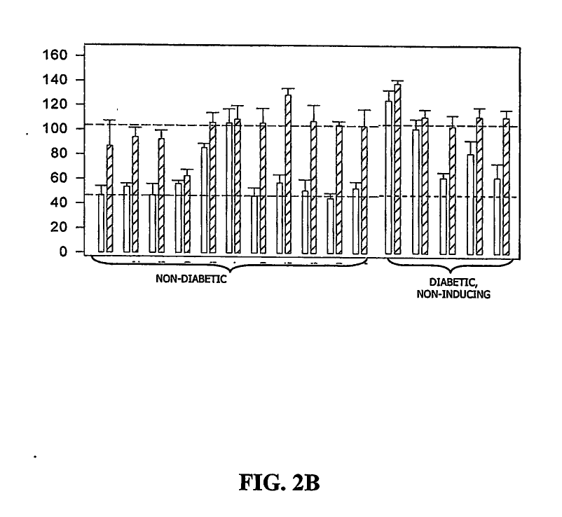

Anterior chamber fluids were collected from diabetic patients and normal volunteers at the time of diagnosis. After 5 years of follow-up the patients were segregated in 2 groups that did or did not develop proliferative retinopathy (+PR and −PR respectively). Frozen samples were then analyzed for PEDF and VEGF content (FIG. 1) and for angiogenic activity (FIG. 2). VEGF increase was less than 2-fold between control and −PR diabetic groups and not significantly altered after progression to +PR. PEDF decreased 2-fold between controls and −PR and further ≧4-fold after transition to +PR. Thus, higher VEGF to PEDF ratios at the onset were typical in the +PR group and predictive of impaired vision later in the course of disease progression.

Higher VEGF/PEDF ratios correlated with increased angiogenic activity in the ocular fluid. The majority of samples collected from control or −PR cohorts of patients were neutral in the in vitro angiogenesis assay while on the average the samples from +PR cohorts were angiogenic (FIG. 2A). In the samples that were non-angiogenic, inducing activity could be unmasked with anti-PEDF neutralizing antibodies (FIG. 2B), indicating that PEDF is one of the key factors responsible for the normal, avascular state of the retina and the vitreous.

Diabetic patients showing higher VEGF/PEDF ratios are candidates for prophylactic treatment with an anti-angiogenic PEDF fragment or analog thereof to delay or prevent onset of proliferative retinopathy.

### Example 2

### Distinct Peptides Responsible for Angio-Inhibitory and Neurotrophic Functions of PEDF

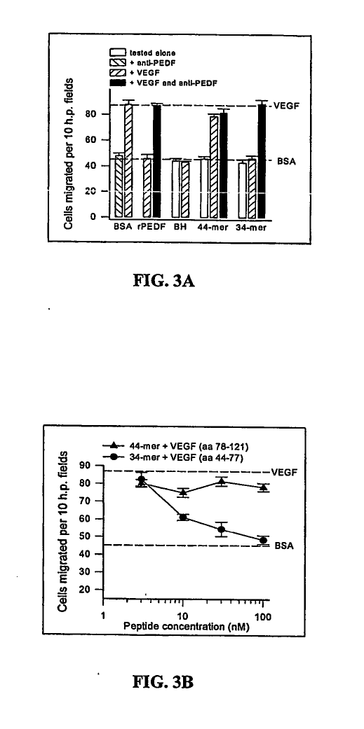

Two large peptides from PEDF, a “34-mer” (amino acids 24-57 of SEQ ID NO:1) and a “44-mer” (residues 58-101 of SEQ ID NO:1), were tested for their ability to block angiogenesis. The 44-mer has been previously shown to bind to and induce differentiation in Y-79 retinoblastoma cells. (Alberdi et al., 1999) In the anti-angiogenesis assays, only the 34-mer inhibited VEGF-induced angiogenesis in vitro. Anti-angiogenic activity of the 34-mer peptide was blocked with neutralizing antibodies effective against the whole molecule. The 34mer inhibited VEGF-induced endothelial cell chemotaxis at 100 nM with an ED₅₀ of ˜6 nM, while the 44-mer showed no inhibitory activity at 100 nM or higher (FIG. 3).

The 34-mer peptide induced apoptosis of cultured endothelial cells with maximal effect reached at doses that also blocked endothelial cell chemotaxis (100 nM, with an ED₅₀ of ˜3 nM). Apoptosis by PEDF and by the 34-mer were mediated by the same signaling events including activation of JNK-1. Apoptosis by both PEDF and by its 34-mer fragment were abolished in the presence of JNK-1 specific inhibitor SP600125 (BioMol). 50-100 nM SP600125 reduced apoptosis by the 34-mer to the background levels. The 44-mer had no activity. The data demonstrate that the neurotrophic and anti-angiogenic functional surfaces of PEDF are spatially distinct.

Cell Culture

Human umbilical vein endothelial cells (HUVECs, VEC Technologies), between passages 3 and 12, are cultured on 0.01%-gelatinized surfaces at 5% CO2 in basal endothelial cell medium (MCDB131, Sigma, St. Louis, Mo.) complemented with EC growth supplements (Bio Whittacker). The cells are grown to confluence and passed at a dilution 1:4.

Boyden Chamber Migration

HUVEC migration assay are performed as previously described (Good et al., PNAS, 1990). The cells are starved overnight in MCDB131 medium supplemented with 0.1% bovine serum albumin (BSA, Sigma) and placed at 1.5 10⁶ cells/ml in the bottom part of a 48-well modified Boyden chambers (Neuroprob Corp.), separated from the top part by gelatinized micro porous membrane (8 μm pore size, Nucleopor/Whatman). The inverted chambers are incubated for 1.5 h for the cells to attach. The chamber are then re-inverted, test substances added to the top part of the top well and incubated for additional 3h30 to allow migration. The chamber are disassembled, the membranes fixed, the cells visualized using Diff-Quick staining kit (Fisher). Then, the stained membranes are dried, mounted, and the cells migrated to the top part of membrane counted in 10 high-powered (400×) fields. MCDB131 containing 0.1% BSA, is used as a negative control and 10 ng/ml bFGF as a positive control. Each substance is tested in quadruplicate to allow statistical evaluation of the data within a single experiment. Each experiment is repeated 3 times to ensure the reproducibility.

Apoptosis Assay

Cells were plated on gelatinized glass cover slips in 24-well tissue culture plates at 5×10⁴ cells/well, treated with indicated compounds in low serum (0.2%), fixed in 1% buffered paraformaldehyde, stained using the ApopTag in situ cell death detection kit according to the manufacturer's instructions (Serologicals corporation) and counterstained with propidium iodide. The percentage of terminal deoxynucleotidyl transferase-mediated dUTP nick end labeling (TUNEL)-positive cells was calculated as the number of TUNEL-positive cells (counted in 2-6 randomly selected fields from two different chambers) divided by the total number of cells. 600-1200 cells were scored for each treatment.

### Example 3

### Preparation of PEDF Peptides

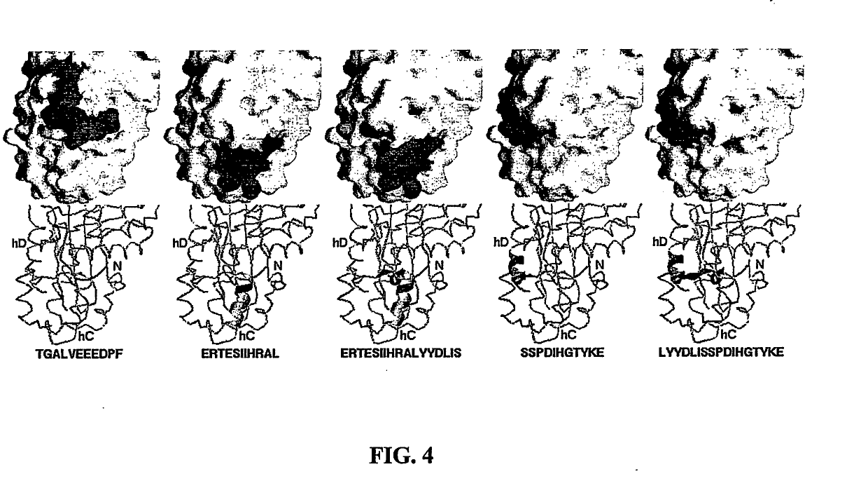

Inspection of the three-dimensional structure of PEDF (See Simonovic et al., 2001) reveals two sections of backbone that represent the middle and bottom patches of a highly acidic region. They are the amino terminus, and the hC-loop-hD section. These sections could either contribute to one larger discontinuous epitope, or have independent functional roles. Both possibilities were tested though the design of a series of PEDF peptides. The peptides intended to separately mimic parts of these sections are described in Table 1 and FIG. 4.

TABLE 1

First Generation of PEDF Peptides from Amino Terminus and hC-loop-hD Section

| Sequence | Residue | | | Name |
|---|---|---|---|---|
| N-Terminus hC loop hD | Range | pI | MW | (SEQ ID NO) |
| acetyl-TGALVEEEDPF-amide | 16-26 | 3.5 | 1248.3 | TGA |
| | | | | (SEQ ID NO: 2) |
| acetyl-ERTESIIHRAL-amide | 78-88 | 6.9 | 1366.5 | ERT-S |
| | | | | (SEQ ID NO: 3) |
| acetyl-ERTESIIHRALYYDLIS-amide | 78-94 | 5.5 | 2121.3 | ERT-L |
| | | | | (SEQ ID NO: 4) |
| acetyl-SSPDIHGTYKE-amide | 94-104 | 5.5 | 1275.3 | SSP |
| | | | | (SEQ ID NO: 5) |
| acetyl-LYYDLISSPDIHGTYKE-amide | 88-104 | 4.8 | 2056.2 | LYY |
| | | | | (SEQ ID NO: 6) |

The rationale for the design of these five peptides was as follows. The amino terminal peptide was intended to separately represent the middle patch of the entire acidic region, presenting the E₂₁EED string of amino acids as a folded unit. The remaining four constructs were intended to dissect the bottom patch of the region, composed of hC-loop-hD.

The peptides described above were made to order by the Research Resource Center (RRC) at the University of Illinois at Chicago. They were prepared to 95% purity as determined by mass spectrometry. With the terminal acetylation and amidylation modifications, the α-helices were expected to retain their secondary structure. The peptides were soluble in the in vitro assay buffer.

The peptides were tested in vitro for their ability to block endothelial cell migration up the gradient of pro-angiogenic bFGF. Two of the five peptides, named TGA and ERT-L, showed anti-angiogenic activity in this assay.

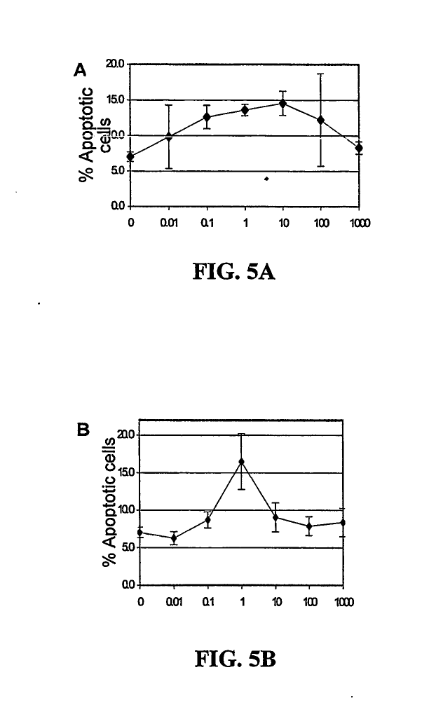

The two peptides that showed anti-angiogenic activity in the endothelial chemotaxis assay, TGA and ERT-L, were assayed for apoptosis and corneal anti-angiogenesis. Human umbilical vein endothelial cells (HUVEC, NCI) were grown to 90% confluence and treated with peptides overnight in low (0.2%) serum. Apoptotic cells were detected using TUNEL assay kit (InterGene) and percent apoptotic cells were calculated (FIG. 5).

The TGA and ERT-L peptides that showed inhibitory effects in the endothelial cell chemotaxis assay and induced apoptosis in vitro also blocked mouse corneal neovascularization in vivo, while the remaining peptides were neutral (Table 2). Angiogenesis was induced in the mouse cornea by implanting slow release pellets containing bFGF (50 ng/pellet). Peptides at 10 and 100 μM were added where indicated. Angiogenesis was observed on day 5 after implantation and a vigorous ingrowth of the blood vessels reaching the pellet was scored as a positive response. The peptide abbreviations are as above; PBS is phosphate buffer solution.

TABLE 2

| | | | Positive |
|---|---|---|---|
| Compound added | Concentration | bFGF | comeas/total |
| 1. PBS | — | + | 4/4 |
| 2. LYY | 10 μM | + | 5/6 |
| | 100 μM | + | 5/5 |
| 3. SSP | 10 μM | + | 6/6 |
| | 100 μM | + | 4/4 |
| 4. TGA | 10 μM | + | 4/6 |
| | 100 μM | + | 2/6 |
| 5. ERT-S | 10 μM | + | 5/6 |
| | 100 μM | + | 4/6 |
| 6. ERT-L | 10 μM | + | 2/6 |
| | 100 μM | + | 1/5 |

### Example 4

### Production of PEDF Variants

The three-dimensional structure of the PEDF molecule was determined by x-ray diffraction methods. (Simonovic et al., 2001) The structural results have been analyzed in terms of 1) charge distribution, 2) earlier PEDF studies, (Alberdi et al., 1998; Alberdi et al., 1999; and Kostanyan et al., 2000) and 3) regions of the molecule with functional relationships to serpins. The most likely signaling surfaces of the PEDF molecule have been selected for modeling.

We identified three peptide fragments of PEDF that have anti-angiogenic activity. They are all clustered in the amino terminal portion of the PEDF sequence, between residues 16 and 94. They are 1) the 34-mer peptide, 2) the TGA peptide, and 3) the ERT-L peptide (FIG. 15). Another peptide known as the 44-mer was previously shown to have neurotrophic activity, (Alberdi et al., 1999) but does not have anti-angiogenic activity.

The results above place the anti-angiogenic signaling surface of PEDF on the acidic patches of the molecule. To extend these studies and confirm the peptide results, PEDF variants were made with mutations in those regions to be tested for anti-angiogenic activity. Two variants have acidic to serine changes for two sets of residues: one in the bottom patch including α-helix C, and the other in the top patch, involving α-helix H and β-strand 1B. They are:

Variant 1: D77/E78/E81 to S77/S78/S81

Variant 2: D236/D238/D280/E284 to S236/S238/S280/S284

The electrostatic effect of these changes is to individually neutralize the bottom and top acidic patches. They also alter the molecular topology in those regions.

The variants were made using the Quick-Change PCR protocol. BHK cells were transfected with either variant 1 or 2 along with the drug resistance plasmids. (Stratikos et al., 1996) Transient expression was seen by western blotting of media with an anti-PEDF monoclonal antibody (Chemicon, Temecula, Calif.).

Transfected cells are selected for drug resistance in order to produce stable cell lines expressing the PEDF variants. Overexpression experiments of the variants are performed. After overexpression, the variants are assayed as above.

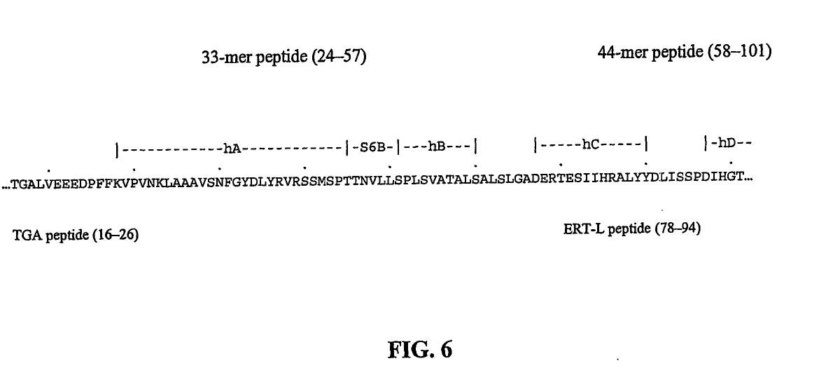

Study of FIG. 6 shows that these structure/function results require careful interpretation. Firstly, the TGA and 34-mer peptides overlap by only three residues (PFF), so either the active region is those three residues, or each of the peptides only partially covers the signaling surface. Secondly, the active ERT-L peptide is separate from the active TGA and 34-mer peptides, so the signaling surface likely is composed of linearly discontinuous but spatially clustered parts of the molecule, i.e., discontinuous functional epitopes. The spatial proximity of the 34-mer, TGA, and ERT-L is confirmed by the three-dimensional structure. Therefore, each of these three peptides contributes partially to the signaling surface. The third and final interpretation is more problematic: the ERT-L peptide is active, but the 44-mer peptide is inactive, and ERT-L is contained entirely within the 44-mer. This situation could arise through two possible scenarios: either ERT-L gave a false positive on the anti-angiogenesis assay, or the 44mer gave a false negative. We favor the latter interpretation, because ERT-L is a shorter 17-residue peptide composed of α-helix C, whereas the 44-mer is a very long peptide with many hydrophobic, internal residues and multiple secondary structure components. It is likely that ERT-L would more readily assume a soluble, regular conformation in solution than the 44-mer.

Shorter peptides can be synthesized to dissect the activities of the TGA, 34mer, and ERT-L peptides, and also peptides that will overlap some of the three active sequences while still observing the borders of secondary structure elements of the full PEDF molecule.

Table 3 lists 20 representative peptides chosen for assay for anti-angiogenic activity. Since the purpose for choosing each type of peptide is given in the table, it is appropriate here to summarize the rationale behind the different categories of experiments.

TABLE 3

Examples of Additional PEDF Peptides

| | |
|---|---|
| Sequence^{a} | Purpose |

| | |
|---|---|
| VEEDP | TGA fragment |
| TGALV(QQQ)DPF | Varieties of mu- |
| | tated TGA |
| TGALVEEEDPFFKVPVNK | Extended TGA |
| EEEDPFFKVPVNK | TGA/34-mer frag- |
| | ment |
| TGASSEEEDP | Improved solubil- |
| | ity of TGA |
| PVNKLAAAVSNFGYDLYRVRSSMSP | hA fragment of the |
| | 34-mer |
| PVNKLAAAVSNFGYNLYRVRSSMSP | Mutated hA |
| KVPVNK | hA fragment |
| SNFGYD | hA fragment |
| YRVRSSMSP | hA fragment |
| DERTES | ERT-L fragment |
| HRALYYD | ERT-L fragment |
| YYDLIS | ERT-L vs. ERT-S |
| ERTESIIHRALYYNLIS | ERT-L vs. ERT-S |
| ERTESSSHRALYYDSSS | Improved solubil- |
| | ity of ERT-L |
| (Q)RT(Q)SIIHRALYY(N)LIS | Varieties of mu- |
| | tated ERT-L |
| ERTESIIHRALYYDLISSPDIHGTYKELLD | hC-loop-hD |
| TGA ± 34-mer ± ERT-L | Synergistic ef- |
| | fects of active |
| | peptide mixtures |
| DERTESIIHRALYYDNNKVPVNKLAAAVSNFG | Permutation/liga- |
| | tion of hC and hA |
| TQVEHR | Gettins et al. |
| | (1996) |

^{a}All peptides will have acetyl groups on their amino terminus and amide groups on their carboxy terminus

The designs of these peptides reflect a variety of approaches. To begin, some peptides are logical progressions from the active TGA, 34-mer, and ERT-L peptides (e.g.,: shorter fragments). Examples are the VEEDP fragment of TGA, the KVPVNK and other fragments of the A helix of the 34-mer, the DERTES and other fragments of ERT-L, etc.

A simple extension of the basic design is to mutate specific residues in the active peptides and test for loss of function, in order to pinpoint the functional group(s). The preferred groups to mutate first are the acidic residues, e.g., any of the three glutamates in TGA, singly or in combination; D44 of the 34mer; E78, E81, or D91 of ERT-L, singly or in combination; etc.

Another approach involves expansion of peptide size. The fact that multiple peptides have activity indicates that the functional surface is larger than any individual peptide. For instance, two sections of PEDF constitute the middle patch of the highly acidic region: the amino terminus, and the 34-mer. These sections could contribute to one larger epitope, so their linear combination many yield greater activity. In this same sense, consider the hC-loop-hD. The hC-loop fragment showed activity, but hD alone did not. Since the two helices are consecutive in the PEDF backbone, there is a chance that the presence of hD could stabilize the connecting loop, increasing activity. Two additional constructs of the helices were made with the intervening loop attached. Additional peptides include the entire hC-loop-hD section.

One way of assaying noncontiguous peptides without increasing peptide length is simply to assay a mix of the original peptides together and test for synergistic effects. In a preferred embodiment, the four possible combinations of TGA, the 34-mer, and ERT-L are assayed. This can be extended in later generations' peptides that show activity.

An important parameter in peptide activity is solubility. All peptides described above were taken directly from the surface of the PEDF molecule, so they have amphiphilic properties required for the protein's folding. To increase their solubility, a number of peptide mutants were designed to remove the hydrophobic components of their amphiphilicities. Examples of this approach are the TGASSEEEDP and other peptides in Table 3.

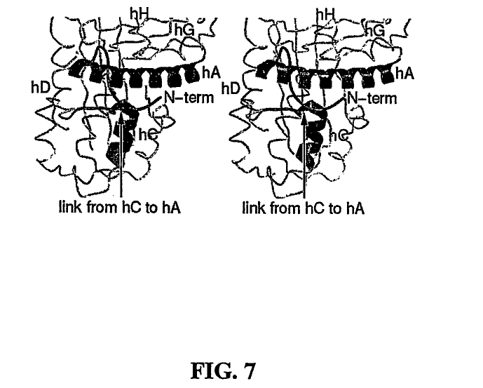

A preferred approach is to combine noncontiguous peptides into new sequential arrangements. An example of this is illustrated in FIG. 7. Helices hA and hC both possess anti-angiogenic activity, so one peptide containing both would likely have greater activity. But the natural peptide from the original PEDF sequence with the intervening 24 residues containing s6B, hB, and turns would be large and hydrophobic (see FIG. 6). However, note the spatial relationships between the two helices: they are close together, and they are oriented such that the carboxyl terminus of hC is proximal to the amino terminus of hA. A peptide linker spanning that short space would create the new sequence hC-N-term-hA that may have greater activity because it would contain all the functional groups of the N-term, hA, and hC in a smaller peptide with the correct spatial arrangement. The nature of the linker may be critical, so a variety of residues can be tested. This permutation and ligation approach has applications in other situations.

Additional peptides correspond to helices G, H, and s1B, along with peptides representing β-strand 5 of β-sheet A of the PEDF molecule, an entirely separate region that has recently been implicated in activity. (Kostanyan et al., 2000)

The peptides can be made with solid phase methods using fmoc chemistry on a Ranier Symphony Synthesizer, followed by purification on HPLC and validation by mass spectrometry. Peptides that show activity can have their conformations analyzed through NMR spectroscopy. Knowledge of the secondary structure of the active peptides can be used in assessing their activities relative to the intact PEDF molecule. For peptides that show secondary structure in solution, a conformation similar to that in the PEDF molecule would be an important verification of the activity results, and could also suggest structural improvements to further stabilize the peptides. NMR spectra can be measured with Bruker AVANCE-500 or a DRX600. The molecular weights of the peptides are well within the practical limit of feasibility, so their structural determinations would be straightforward with ¹H and natural abundance ¹³C NMR.

If solubility problems occur, new constructs can be made by replacing hydrophobic residues on the interior surface of the amphiphilic α-helices with more soluble side chains while retaining a high propensity for helix formation. Any future solubility problems can be treated with a number of approaches, such as additional solvents (e.g., DMSO), shorter sequences, substitution of hydrophobic residues, alternate modifications of the amino and carboxyl termini, etc.

In a preferred embodiment, peptide solubility is determined before committing to the assays. Solubility may be maximized by restricting peptide length, and designing in hydrophilic groups where necessary.

For peptides that are expected to show activity but do not, one can recheck the composition and sequences of the peptides to assure they are correct. Alternatively, one can reproduce the assays with known active compounds in order to check the protocols.

The results of methods described herein can be used to design and test additional generations of peptides, to select those with highest activity for assay in ischemic retinopathy, to design small molecule mimics, and to initiate receptor labeling and isolation.

It is possible that some peptides may show biological activity in vitro, but still may not have activity in the in vivo assays due to susceptibility to endogenous proteinases. A preferred way to circumvent this problem is by using retro-enantiomers. The retro-enantio concept relies on the observations that a peptide made of D-amino acids in the reverse sequence of the desired peptide will have the same topology but be resistant to proteolysis. This approach has proved successful in a number of unrelated peptide mimic studies. (Jameson et al., 1994; Guichard et al., 1994; and Merrifield et al., 1995) Here, the retro-enantiomer peptide mimics can be designed based on any of the L-peptides that showed in vitro biological activity. They can be tested with the same assays as for the L-peptides. It is important to note that the potencies of anti-angiogenic peptides designed from thrombospondin-1 were increased by two to three orders of magnitude through individual D-amino acid substitutions in an otherwise L-amino acid molecule. (Dawson et al., 1999)

An alternative embodiment to increased stability and bioavailability of designed anti-angiogenics is to reproduce the active agent in the form of peptidomimetics such as peptoids. Peptoids are oligomers of N-substituted glycines that are metabolically stable and as synthetically accessible as peptides. (Simon et al., 1992) Peptoids have been made available in combinatorial libraries for screening in drug discovery. (Zuckermann et al., 1994) Laboratories routinely synthesize peptoids in the same quantity, purity, and price as peptides. Preferably, the design and test of peptoid mimetics is pursued once the peptides with greatest anti-angiogenic activity are identified

### Example 5

### Peptides and Analogs as Anti-Angiogenic Agents in Treatment of Ischemic Retinopathy

Active peptides are tested in a mouse model of the retinopathy of prematurity (ROP). Those yielding promising results are further stabilized by chemical modification and repeatedly tested in the same model. Stabilization and analog development are discussed above. A number of strategies can be employed, including retro-enantiomer design and synthesis and peptoid screening.

Active peptides are ranked in order of their activity in the in vitro migration and apoptosis assay (e.g., using ED₅₀ as a defining characteristic for the ranking). Those with the lowest ED₅₀'s are tested in the corneal angiogenesis assay for the ability to block angiogenesis in vivo when applied locally and systemically. Finally, the most efficacious peptides are tested for the ability to block retinal neovascularization in the mouse ROP model. (Stellmach et al., 2001)

First, peptides are incorporated in a Hydron-Sucralfate slow release pellet. The peptides are tested at doses ranging from 3×, 10×, 30×, 100× and 300× of minimal effective dose determined in migration assay to account for the diffusion rates in the cornea, (Tolsma et al., 1993) in the presence of standard angiogenic stimuli, bFGF and VEGF. The extent of neovascularization can be characterized in at least two ways:
qualitatively, as the number of positive corneas of total implanted (% positive responses). One can score corneas with numerous vessels reaching into the pellet as positive (+), those with fewer vessels that fail to reach the pellet as weak positive (±), and those with no more then few occasional vessels not reaching the pellet, as negative (−).
semi-quantitatively, as the total length of capillary sprouts from the limbus in the direction of the pellet. The length is determined using computer analysis of the digital images of the corneas (modified Corel Tracer software).

Dose-response curves of inhibition are generated for each peptide and ED₅₀'s as well as minimal effective doses determined and compared. Each peptide concentration is tested in a minimum of 5 eyes and the results subjected to statistical evaluation.

Of the peptides tested locally, the most potent ones are selected and applied systemically in the corneal neovascularization assay. Mice are given corneal implants containing bFGF or VEGF and treated with intraperitoneal injections of the test peptide(s) or vehicle control. For the peptide treatment, the doses are calculated based on the average animal weight of 20 g, so that the concentrations range from 3×, 10×, 30× and 100× and 300× from the minimally effective concentration determined in vitro, to account for the rapid degradation in the bloodstream. The results are evaluated as above and the best ones tested in the mouse ROP model.

ROP experiments are carried out as is standardly known. (Connolly et al., 1988; Smith et al., 1994; and Stellmach et al, 2001) Female C57/Bl6J mice with neonates are placed in hyperoxia chamber (75% O₂:25% N₂) from postnatal day 7 (P7) to P12, then removed to room air and given intraperitoneal injections of peptide or vehicle control (PBS) daily from P12 through P16, with doses within the range determined previously, in corneal neovascularization assays (see above). Each dose will is tested in 4-5 mouse pups. At P17 the pups are weighed, sacrificed, the eyes extracted, snap-frozen in OCT compound and sectioned in the plane parallel to the optical nerve; Cryosections are stained for the endothelial marker CD31 using rat-anti-mouse polyclonal antibodies and Texas-Red conjugated goat anti-rat secondary antibody. To visualize the retinal cell layer, the sections are counterstained with DAPI to highlight all the nuclei. Digital fluorescent images are taken and the number of CD31-positive structures in each eye determined in 4 random high-powered fields using MetaView software package. The data are presented as averages with S.E.M. and statistically evaluated with paired Student's T-test. Pups that remained under normoxic conditions for the duration of the experiment are used for comparison. Pups treated with vehicle PBS and inactive peptide are used as a negative control. Purified, recombinant PEDF serves as a positive control.

The peptide or peptides that showed anti-angiogenic activity when given systemically in the corneal angiogenesis assay will be effective in the ROP model and cause a decrease in the number of aberrant vessels, leakage and retinal detachment. Although most of the active peptides fit into the same region within the ligand-binding domain of the putative PEDF receptor, it is not impossible that some of the shorter peptides bind their own characteristic spots within the ligand-binding domain. Such peptides might be complementary and may have additive, if not synergistic effects in suppressing angiogenesis. Potential candidates could be determined by additional binding studies and tested in concert.

In alternative embodiments, the stability and toxicity of the active peptides and mimics are determined and tested in other models of angiogenesis-dependent eye disease, including the laser model of macular degeneration. (Mori et al., 2001 and Kaplan et al., 1999).

An example of another useful in vivo assay is the chick chorioallantoic membrane assay (CAM). It may be used to determine whether a PEDF fragment or analog thereof is capable of inhibiting neovascularization in vivo. Taylor and Folkman, 1982, Nature (London) 297:307-312. The effect of troponin a PEDF fragment or analog thereof on growing embryonic vessels is studied using chick embryos in which capillaries appear in the yolk sac at 48 h and grow rapidly over the next 6-8 days.

Three day post fertilization chick embryos are removed from their shells and placed in plastic petri dishes (1005, Falcon). The specimens are maintained in humidified 5% CO.sub.2 at 37.degree. C. On day 6 of development, samples of purified PEDF fragment or analog thereof are mixed in methylcellulose disks and applied to the surfaces of the growing CAMs above the dense subectodermal plexus. Control specimens in which CAMs are implanted with empty methylcellulose disks are also prepared. The CAMs are injected intravascularly with India ink/Liposyn to more clearly delineate CAM vascularity. Taylor et al., 1982, Nature 297:307-312.

Following a 48 hour exposure of the CAMs to the PEDF fragment or analog thereof, the area around the implant is observed and evaluated. Test specimens having avascular zones completely free of India-ink filled capillaries surrounding the test implant indicate the presence of an inhibitor of embryonic neovascularization. In contrast, the control specimens show neovascularization in close proximity or in contact with the methylcellulose disks.

Histological mesodermal studies are preformed on the CAMs of test and control specimens. The specimens are embedded in JB-4 plastic (Polysciences) at 4.degree. C. and 3.mu.m sections are cut using a Reichert 2050 microtome. Sections are stained with toluidine blue and micrographs are taken on a Zeiss photomicroscope using Kodak™ times.100 and a green filter.

Yet another useful in vivo assay is the rabbit corneal pocket assay. Male NZW rabbits weighing 4-5 lbs. are anesthetized with intravenous pentobarbital (25 mg/kg) and 2% xylocalne solution is applied to the cornea. The eye is proptosed and rinsed intermittently with Ringer's solution to prevent drying. The adult rabbit cornea has a diameter of approximately 12 mm. An intracorneal pocket is made by an incision approximately 0.15 mm deep and 1.5 mm long in the center of the cornea with a No. 11 scalpel blade, using aseptic technique. A 5 mm-long pocket is formed within the corneal stroma by inserting a 1.5 mm wide, malleable iris spatula. In the majority of animals, the end of the corneal pocket is extended to within 1 mm of the corneal-scleral junction. In a smaller series of 22 rabbits implanted with tumor alone, pockets are placed at greater distances×2-6 mm from the corneal-scleral junction by starting the incision away from the center.

In the first assay, polymer pellets of ethylene vinyl acetate (EVAc) copolymer are impregnated with test substance and surgically implanted in a pocket in the rabbit cornea approximately 1 mm from the limbus. When this assay system is being used to test for angiogenesis inhibitors, either a piece of V2 carcinoma or some other angiogenic stimulant is implanted distal to the polymer, 2 mm from the limbus. On the opposite eye of each rabbit, control polymer pellets that are empty are implanted next to angiogenic stimulant in the same way. In these control corneas, capillary blood vessels start growing towards the tumor implant in 5-6 days, eventually sweeping over the blank polymer. In test corneas, the directional growth of new capillaries from the limbal blood vessels towards the tumor occurs at a reduced rate and is often inhibited such that an avascular region around the polymer is observed. This assay is quantitated by measurement of the maximum vessel lengths with a stereoscopic microscope.

### LITERATURE CITED

Aiello, L. P., Avery, R. L., Arrigg, P. G., Keyt, B. A., Jampel, H. D., Shah, S. T., Pasquale, L. R, Thieme, H., Iwamoto, M. A., Park, J. E., Nguyen, H. V., Aiello, L. M., Ferrara, N., and King, G. C. (1994) Vascular endothelial growth factor in ocular fluid of patients with diabetic retinopathy and other retinal disorders. N. Engl. J. Med. 331, 1480-1487.
Alberdi, E., Aymerich, M. S., and Becerra, S. P. (1999) Binding of pigment epithelium-derived factor (PEDF) to retinoblastoma cells and cerebellar granule neurons. Evidence for a PEDF receptor. J. Biol. Chem. 274, 31605-31612.
Alberdi, E., Hyde, C. C., and Becerra, S. P. (1998) Pigment epithelium-derived factor (PEDF) binds to glycosaminoglycans: Analysis of the binding site. Biochemistry 37, 10643-10652.
Aymerich, M. S., Alberdi, E. M., Martinez, A., and Becerra, S. P. (2001) Evidence for pigment epithelium-derived factor receptors in the neural retina. Invest. Ophthalmol. Vis. Sci. 42, 3287-3293.
Becerra, S. P., Sagasti, A., Spinella, P., and Notario, V. (1995) Pigment epithelium-derived factor behaves like a noninhibitory serpin. J. Biol. Chem. 270, 25992-25999.
Casey, R., and Li, W. W. (1997) Factors controlling ocular angiogenesis. Am. J. Opthamol. 124, 521-529.
Cayouette, M., Smith, S. B., Becerra, S. P., and Gravel, C. (1999) Pigment epithelium-derived factor delays the death of photoreceptors in mouse models of inherited retinal degenerations. Neurobiol. Dis. 6, 523-532.
Chader, G. (2001) PEDF: Raising both hopes and questions in controlling angiogenesis. Proc. Natl. Acad. Sci. USA 98, 2122-2124.
Chao, J., Miao, R. Q., and Chao, L. Kallistatin: A new angiogenesis inhibitor. 3^{rd} International Symposium on Serpin Biology, Structure, and Function. Chicago, Ill., 2002.
Chung, J, Gao, A. G., and Frazier, W. A. (1997) Thrombspondin acts via integrin-associated protein to activate the platelet integrin α_{IIb}β₃. J. Biol. Chem. 272, 14740-14746.
Connolly, S. E., Hores, T. A., Smith, L. E., and D'Amore, P. A. (1988) Characterization of vascular development in the mouse retina. Microvasc. Res. 36, 275-290.
Dawson, D. W., Volpert, 0. V., Gillis, P., Crawford, S. E., Xu, H., Benedict, W., and Bouck, N. P. (1999) Pigment epithelium-derived factor: A potent inhibitor of angiogenesis. Science 285, 245-248.
Dawson, D. W., Volpert, O. V., Pearce, S. F., Schneider, A. J., Silverstein, R. L., Henkin, J., and Bouck, N. P. (1999) Three distinct D-amino acid substitutions confer potent antiangiogenic activity on an inactive peptide derived from a thrombospondin-1 type 1 repeat. Mol. Pharmacol. 55, 332-338.
DeCoster, M. A., Schabelman, E., Tombran-Tink, J., and Bazan, N. G. (1999) Neuroprotection by pigment epithelial-derived factor against glutamate toxicity in developing primary hippocampal neurons. J. Neurosci. Res. 56, 604-610.
Doggett, D. L., Rotenberg, M. O., Pignolo, R. J., Phillips, P. D., and Cristofalo, V. J. (1992) Differential gene expression between young and senescent, quiescent WI-38 cells. Mech. Ageing Dev. 65, 239-255.
Gao, G., Li, Y., Gee, S., Dudley, A., Fant, J., Crosson, C., and Ma, J. X. (2002) Down-regulation of vascular endothelial growth factor and up-regulation of pigment epithelium-derived factor: a possible mechanism for the anti-angiogenic activity of plasminogen kringle 5. J. Biol. Chem. 277, 9492-9497.
Gettins, P. G. W., Patston, P. A., and Olson S. T. (1996) Serpins: Structure, Function, and Biology. R. G. Landes Company, Austin, Tex.
Good, D. J., Polyerini, P. J., Rastinejad, F., Le Beau, M. M., Lemons, R. S., Frazier, W. A., and Bouck, N. P. (1990) A tumor suppressor-dependent inhibitor of angiogenesis is immunologically and functionally indistinguishable from a fragment of thrombospondin. Proc. Natl. Acad. Sci. USA 87, 6624-6628.
Guichard, G., Benkirane, N., Zeder-Lutz, G., van Regenmortel, M. H., Briand, J. P., and Muller, S. (1994) Antigenic mimicry of natural L-peptides with retro-inverso-peptidomimetics. Proc. Natl. Acad. Sci. USA 91, 9765-9769.
Irving, J., Pike, R. N., Lesk, A. M., and Whisstock, J. C. (2000) Phylogeny of the serpin superfamily: Implications of patterns of amino acid conservation for structure and function. Genome Res. 10, 1845-1864.
Jablonski, M. M., Tombran-Tink, J., Mrazek, D. A., and Iannaccone, A. (2000) Pigment epithelium-derived factor supports normal development of photoreceptor neurons and opsin expression after retinal pigment epithelium removal. J. Neurosci. 20, 7149-7157.
Jameson, McDonnell, J. M., Marini, J. C., and Korngold, R. (1994) A rationally designed CD4 analogue inhibits experimental allergic encephalomyelitis. Nature 368, 744-746.
Jiménez, B., Volpert, O. V., Crawford, S. E., Febbraio, M., Silverstein, R. L., and Bouck, N. (2000) Signals leading to apoptosis-dependent inhibition of neovascularization by thrombospondin-1. Nature Med. 6, 41-48.
Kaplan, H. J., Leibole, M. A., Tezel, T., and Ferguson, T. A. (1999) Fas ligand (CD95 ligand) controls angiogenesis beneath the retina. Nature Med. 5, 292-297.
Kenyon, B. M., Voest, E. E., Chen, C. C., Flynn, E., Folkman, J., and D'Amato, R. J. (1996) A model of angiogenesis in the mouse cornea. Invest. Ophthalmol. Vis. Sci. 37, 1625-1632.
King, G., and Suzuma, K. (2000) Pigment-epithelium-derived factor: A key coordinator of retinal neuronal and vascular functions. N. Engl. J. Med. 342, 349-351.
Kostanyan, I. A., Zhokhov, S. S., Astapova, M. V., Dranitsyna, S. M., Bogachuk, A. P., Baidakova, L. K., Rodionov, I. L., Baskin, I. I., Golubeva, O. N., Tombran-Tink, J., and Lipkin, V. M. (2000) The biological function of a fragment of the neurotrophic factor from pigment epithelieum: Structural and functional homology with the differentiation factor of the HL-60 cell line. Bioorg. Khim. 26, 563-570.
Larsson, H., Sjöblom, T., Dixelius, J., Östman, A., Björk, I., and Claesson-Welsh, L. (2000) Antiangiogenic effects of latent antithrombin through perturbed cell-matrix interactions and apoptosis of endothelial cells. Cancer Res. 60, 6723-6729.
Merrifield, R. B., Juvvadi, P., Andreu, D., Ubach, J., Boman, A., and Boman, H. G. (1995) Retro and retroenantio analogs of cecropin-melittin hybrids. Proc. Natl. Acad. Sci. USA 92, 3449-345.
Mori, K., Duh, E., Gehlbach, P., Ando, A., Takahashi, K., Pearlman, J., Yang, H. S., Zack, D. J. Ettyreddy, D., Brough, D. E., Wei, L. L., and Campochiaro, P. A. (2001) Pigment epithelium-derived factor inhibits retinal and choroidal neovascularization. J. Cell Physiol. 188, 253-263.
Mori, K., Duh, E., Gehlbach, P., Ando, A., Takahashi, K., Pearlman, J., Yang, H. S., Zack, D. J., Ettyreddy, D., Brough, D. E., Wei, L. L., and Campochiaro, P. A. (2001) Pigment epithelium-derived factor inhibits retinal and choroidal neovascularization. J. Cell Physiol. 188,253-263.
Nicholls, A., Sharp, K. A., and Honig, B. (1991) Protein folding and association: insights from the interfacial and thermodynamic properties of hydrocarbons. Proteins 11, 281-296.
O'Reilly, M. S., Pirie-Shepherd, S., Lane, W. S., and Folkman, J. (1999) Antiangiogenic activity of the cleaved conformation of the serpin antithrombin. Science 285, 1926-1928.
Ohno-Matsui, K., Morita, I., Tombran-Tink, J., Mrazek, D., Onodera, M., Uetama, T., Hayano, M., Murota, S. I., and Mochizuki, M. (2001) Novel mechanism for age-related macular degeneration: an equilibrium shift between the angiogenesis factors VEGF and PEDF. J. Cell Physiol. 189, 323-333.
Pe'er, J., Folberg, R., Itin, A., Gnessin, H., Hemo, I., and Keshet, E. (1996) Upregulated expression of vascular endothelial growth factor in proliferative diabetic retinopathy. Br. J. Opthamol. 80,241-245.
Pignolo, R. J., Rotenberg, M. O., and Cristofalo, V. J. (1995) Analysis of EPC-1 growth state dependent expression, specificity, and conservation of related sequences. J. Cell Physiol. 162, 110-118.
Shweiki, D., Neeman, M., Itin, A., and Keshet, E. (1995) Induction of vascular endothelial growth factor expression by hypoxia and by glucose deficiency in multicell spheroids: implications for tumor angiogenesis. Proc. Natl. Acad. Sci. USA 92, 768-772.
Simon, R. J., Kania, R. S., Zuckermann, R. N., Huebner, V. D., Jewell, D. A., Banville, S., Ng, S., Wang, L., Rosenberg, S., Marlowe, C. K., Spellmeyer, D. C., Tan, R., Frankl, A. D., Santi, D. V., Cohen, F. E., and Bartlett, P. A. (1992) Peptoids: a modular approach to drug discovery. Proc. Natl. Acad. Sci. USA 89, 9367-9371.
Simonovic, M., Gettins, P. G. W., and Volz, K. (2001) Crystal structure of human PEDF, a potent anti-angiogenic and neurite growth-promoting factor. Proc. Natl. Acad. Sci. USA, 95, 11131-11135.
Smith, L. E., Wesolowski, E., McLellan, A., Kostyk, S. K., D'Amato, R., Sullivan, R., and D'Amore, P. A. (1994) Oxygen-induced retinopathy in the mouse. Invest. Ophthalmol. Vis. Sci. 35, 101-111.
Steele, F. R., Chader, G. J., Johnson, L. V., and Tombran-Tink, J. (1993) Pigment epithelium-derived factor: neurotrophic activity and identification as a member of the serine protease inhibitor gene family. Proc. Natl. Acad. Sci. USA 90, 1526-1530.
Stellmach, V., Crawford, S. E., Zhou, W., and Bouck, N. (2001) Prevention of ischemia-induced retinopathy by the natural ocular antiangiogenic agent pigment epithelium-derived factor. Proc. Natl. Acad. Sci. USA 98, 2593-2597.
Stratikos, E., Alberdi, E., Gettins, P. G. W., and Becerra, S. P. (1996) Recombinant human pigment epithelium-derived factor (PEDF): Characterization of PEDF overexpressed and secreted by eukaryotic cells. Protein Sci. 5, 2575-2582.
Taniwaki, T., Becerra, S. P., Chader, G. J., and Schwartz, J. P. (1995) Pigment epithelium-derived factor is a survival factor for cerebellar granule cells in culture. J. Neurochem. 64, 2509-2517.
Tolsma, S., S., Volpert, O. V., Good, D. J., Frazier, W. A., Polyerini, P. J., and Bouck, N. (1993) Peptides derived from two separate domains of the matrix protein thromobospondin-1 have anti-angiogenic activity. J. Cell Biol. 122, 497-511.
Tombran-Tink, J., and Johnson, L. V. (1989) Neuronal differentiation of retinoblastoma cells induced by medium conditioned by human RPE cells. Invest. Ophthalmol. Vis. Sci. 30, 1700-1707.
Tombran-Tink, J., Chader, G. G., and Johnson, L. V. (1991) PEDF: a pigment epithelium-derived factor with potent neuronal differentiative activity. Exp. Eye Res. 53, 411-414.
Tombran-Tink, J., Mazuruk, K., Rodriguez, I. R., Chung, D., Linker, T., Englander, E., and Chader, G. J. (1996) Organization, evolutionary conservation, expression and unusual Alu density of the human gene for pigment epithelium-derived factor, a unique neurotrophic serpin. Mol. Vis. 2, 11-19.
Volpert, O. V., Zaichuk, T., Zhou, W., Reiher, F., Ferguson, T. A., Stuart, P. M., Amin, M., and Bouck, N. P. (2002) Inducer-stimulated Fas targets activated endothelium for destruction by anti-angiogenic thrombospondin-1 and pigment epithelium-derived factor. Nature Med. 8, 349-357.
Wennogle, L. P., Conder, L., Winter, C., Braunwalder, A., Vlattas, S., Kramer, R., Cioffi, C., and Hu, S. I. (1994) Stabilization of C5a receptor-G-protein interactions through ligand binding. J. Cell Biochem. 55, 380-388.
Yabe, T., Wilson, D., and Schwartz, J. P. (2001) NFκB is required for the neuroprotective effects of pigment epithelium-derived factor (PEDF) on cerebellar granule neurons. J. Biol. Chem. 276, 43313-43319.
Zhang, M., Volpert, O., Shi, Y. H., and Bouck, N. (2000) Maspin is an angiogenesis inhibitor. Nature Med. 6, 196-199.
Zuckermann, R. N., Martin, E. J., Spellmeyer, D. C., Stauber, G. B., Shoemaker, K. R., Kerr, J. M., Figliozzi, G. M., Goff, D. A., Siani, M. A., Simon, R. J., Banville, S. C., Brown, E. G., Wang, L., Richter, L. S., and Moos, W. H. (1994) Discovery of nanomolar ligands for 7-transmembrane G-protein-coupled receptors from a diverse N-(substituted)glycine peptoid library. J. Med. Chem. 37, 2678-2685.

1. An anti-angiogenic pigment epithelium-derived factor (PEDF) fragment, the anti-angiogenic peptide having an amino acid sequence consisting essentially of 5-50 contiguous amino acids of SEQ ID NO:1.

2. The anti-angiogenic PEDF fragment of claim 1 comprising an amino acid sequence selected from the group consisting of:

| | |
|---|---|
| (a) | TGALVEEEDPF; |
| (b) | ERTESIIHRALYYDLIS; |
| (c) | DPFFKVPVNKLAAAVSNFGYDLYRVRSSMSPTTN. |

3. The anti-angiogenic peptide of claim 1, wherein the PEDF fragment comprises an altered terminus.

4. A composition comprising a PEDF fragment of claim 1, and a pharmaceutical buffer or excipient.

5. A method of inhibiting endothelial cell migration or proliferation comprising contacting an endothelial cell with a composition comprising an effective amount of a pigment epithelium-derived factor (PEDF) peptide fragment, wherein the PEDF peptide fragment has anti-angiogenic activity.

6. The method of claim 5, wherein the endothelial cell is contacted in vitro.

7. The method of claim 6, wherein the endothelial cell is contacted in vivo.

8. The method of claim 7, the contacting comprises the step of administering the effective amount PEDF peptide fragment to a patient with a disease or disorder associated with neovascularization.

9. The method of claim 8, wherein the effective amount of PEDF peptide fragment inhibits angiogenesis.

10. The method of claim 9, wherein the disease or disorder associated with neovascularization is an ophthalmologic disease or disorder.

11. The method of claim 9, wherein the disease or disorder associated with neovascularization is a malignant or metastatic condition.

12. The method of claim 7, wherein the composition comprises a pharmaceutical buffer or excipient.

13. (canceled)

14. (canceled)

15. (canceled)

16. A medical device comprising the composition of claim 4.

17. A method of treating cancer comprising administering a therapeutically effective amount of an anti-angiogenic pigment epithelium-derived factor (PEDF) fragment to a patent in need thereof.

18. A method of treating an opthalmological disease of disorder comprising administering a therapeutically effective amount of an anti-angiogenic pigment epithelium-derived factor (PEDF) fragment to a patent in need thereof.

19. A method of predicting whether a diabetic patient will develop proliferative retinopathy comprising determining the ratio of vascular endothelial growth factor (VEGF) to PEDF in an ocular fluid sample from said patient.

20. The method of claims 19, wherein the ratio of VEGF to PEDF is compared to one or more control ratios.

21. An anti-angiogenic PEDF fragment analog comprising one or more amino acid insertions, deletions, or substitutions to a PEDF fragment of claim 1.
